(19) United States
(12) Patent Application Publication
(10) Pub. No.: US 2026/0224375 A1
(43) Pub. Date: Aug. 6, 2026

(54) AN UPPER LIMB PROSTHETIC HAND FOR TRANSRADIAL AMPUTEE

(71) Applicant: LIFE AND LIMB PRIVATE LIMITED, Ghaziabad, Uttar Pradesh (IN)

(72) Inventor: Nishant AGARWAL, Ghaziabad (IN)

(73) Assignee: LIFE AND LIMB PRIVATE LIMITED, Ghaziabad, Uttar Pradesh (IN)

(21) Appl. No.: 19/147,931

(22) PCT Filed: May 18, 2023
(86) PCT No.: PCT/IB2023/055119

§ 371 Date: Jul. 14, 2025

(30) Foreign Application Priority Data
Jan. 14, 2023 (IN) 202311002959

(51) Int. Cl. A61F 2/72; A61F 2/58; A61F 2/76

(52) U.S. Cl. CPC A61F 2/72; A61F 2002/587; A61F 2002/769

(57) ABSTRACT

An upper limb prosthetic hand for transradial amputee is described. The prosthetic hand may include a terminal device comprising a thumb and four fingers; a longitudinally extending hollow forearm shell removably coupled to the terminal device, wherein the hollow forearm shell comprises a coupling means, and wherein the coupling means is to couple the prosthetic hand with a residual stump of the amputee; and a plurality of sensors mounted on an inner surface of the hollow forearm shell at the coupling means, wherein the plurality of sensors are in contact with the residual stump of the amputee. At least one of the plurality of sensors may be configured to: monitor a muscle movement in the residual stump; and based on the monitored muscle movement in the residual stump, generate electrical signals.

### TECHNICAL FIELD

The present disclosure, in general, relates to the field of artificial limbs for amputees and, in particular, discloses an upper limb prosthetic hand for assisting a transradial amputee.

### BACKGROUND

Background description includes information that may be useful in understanding the present invention. It is not an admission that any of the information provided herein is prior art or relevant to the presently claimed invention, or that any publication specifically or implicitly referenced is prior art.

Generally, harness-based hands may be used by various amputees. Available harness-based hands may be bulky and inefficient, thereby possessing a difficulty to the user for assistance. Electro-mechanical prosthetic hands may be available to cater to the requirements of amputees, however, they are costly and may be unable to be properly used by the patients, i.e., amputees.

Myoelectric prosthetic hands are increasingly used in recent times, to cater to the requirements of amputees. However, the conventional and existing myoelectric prosthetic hands may possess a number of problems. The available solutions are bulky and of pre-defined set of sizes. This may be inefficient to be used by the amputees. Even further, various components of the prosthetic hand may be housed at various locations in the hand, thereby providing an extra amount of weight to the hand. As a result, it may require a significant amount of effort on the user's end to use the hand.

Further, the manner in which the thumbs and the fingers of the available prosthetic hands function, may be inefficient. The thumb may be fixed, thereby limiting the use of the prosthetic hands. As a further result of this, the amputee may have difficulty in performing the daily tasks.

There is, therefore, a need to provide a robust, efficient upper limb prosthetic hand to assist the amputees, particularly transradial amputees.

### SUMMARY

Aspects of the present disclosure relate to the field of artificial limbs for amputees and, in particular, discloses an upper limb prosthetic hand for assisting a transradial amputee.

An upper limb prosthetic hand for transradial amputee is described. The prosthetic hand may include a terminal device comprising a thumb and four fingers; a longitudinally extending hollow forearm shell removably coupled to the terminal device, wherein the hollow forearm shell comprises a coupling means, and wherein the coupling means is to couple the prosthetic hand with a residual stump of the amputee; and a plurality of sensors mounted on an inner surface of the hollow forearm shell at the coupling means, wherein the plurality of sensors are in contact with the residual stump of the amputee. At least one of the plurality of sensors may be configured to: monitor a muscle movement in the residual stump; and based on the monitored muscle movement in the residual stump, generate electrical signals.

In another aspect, the upper limb prosthetic hand may further include a plurality of controllers communicatively coupled to the plurality of sensors and the terminal device.

In yet another aspect, at least one of the plurality of controllers may be configured to: based on the generated electrical signals by at least one of the plurality of sensors, cause at least one of the thumb and the four fingers in the terminal device to move.

In yet another aspect, the terminal device is a palm unit.

In yet another aspect, coupling the prosthetic hand with the residual stump of the amputee comprises the longitudinally extending hollow forearm shell to receive a portion of the residual stump of the amputee.

In yet another aspect, the coupling means is one of a liner, a socket, or a combination thereof.

In yet another aspect, the plurality of sensors are surface electromyography (EMG) sensors.

In yet another aspect, the plurality of surface EMG sensors are made of an origami-inspired flexible material.

In yet another aspect, the terminal device further comprises a display unit and an input unit.

In yet another aspect, the input unit is to receive one of a touch-based input, a voice-based input, or a combination thereof, from the amputee.

In yet another aspect, the terminal device further comprises a power source, wherein the power source is to power the prosthetic hand.

In yet another aspect, the power source is at least one of a plurality of Li-ion rechargeable batteries.

In yet another aspect, at least one of the plurality of controllers is implemented in the terminal device.

In yet another aspect, at least one of the plurality of controllers is independently implemented in each of the four fingers of the terminal device.

In yet another aspect, each of the four fingers of the terminal device comprises an independent actuator, and wherein the controller, based on the signals from at least one of the plurality of sensors, is to cause each of the four fingers to move independently.

In yet another aspect, the controller, based on the signals from at least one of the plurality of sensors, is to cause an abduction movement, an adduction movement, a flexion movement, an extension movement, or a combination thereof, in the thumb.

In yet another aspect, the controller may be further configured to cause the terminal device to operate based on an input from a user.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates a perspective view of an exemplary upper limb prosthetic hand, as per an implementation of the present subject matter; and

FIG. 2 illustrates a bottom view of an exemplary terminal device to be implemented in an upper limb prosthetic hand, as per an implementation of the present subject matter.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure.

Exemplary embodiments will now be described more fully hereinafter with reference to the accompanying drawings, in which exemplary embodiments are shown. This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Aspects of the present disclose an upper limb prosthetic hand for assisting a transradial amputee. As would be appreciated, in the proposed prosthetic hand, all the components such as battery, display unit, input unit, etc. may be housed within the terminal device. As a result, the hollow forearm shell may be vacant, thereby able to accommodate a significant number of stump sizes. Further, such prosthetic hand may be convenient to be used by amputee, and may not provide a strain to the amputee.

As would be further appreciated, the terminal device may be removably coupled with the hollow forearm shell, thereby being able to be easily charged. This may further increase the portability of the proposed prosthetic hand.

Furthermore, the thumb of the proposed prosthetic hand may be designed in such a manner that the thumb may be able to perform a variety of movements such as abduction, adduction, flexion, and extension. This may improve the utility of the prosthetic hand. Even further, each of the fingers in the proposed prosthetic hand may include an independent actuator. This may facilitate vibration isolation in the terminal device. This may also facilitate partial hand amputation. All such proposed mechanisms and approaches may aid in assisting the amputee to perform a variety of movement and functionalities.

In addition, the proposed upper limb prosthetic hand may be made based on the size requirements of the user. For example, the other limb of the amputee may be scanned and the prosthetic hand may be made of a similar size. The prosthetic hand may be light-weight as well, thereby not providing any discomfort to the user.

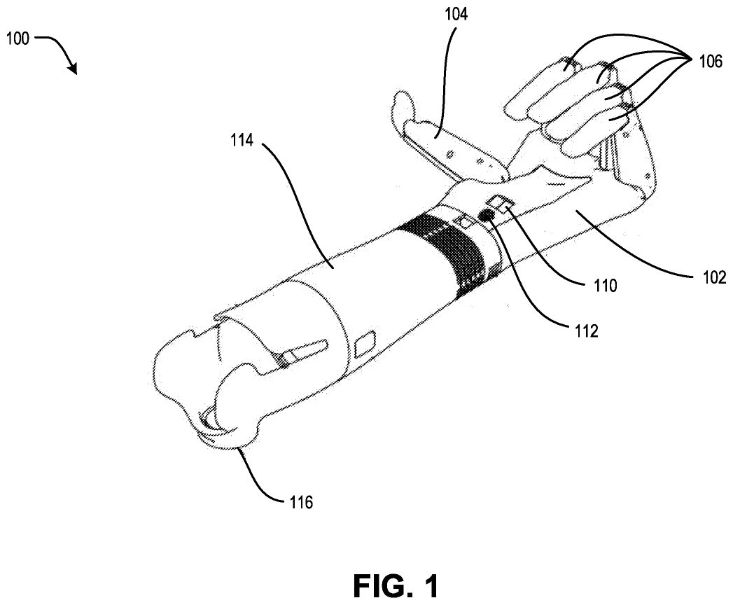

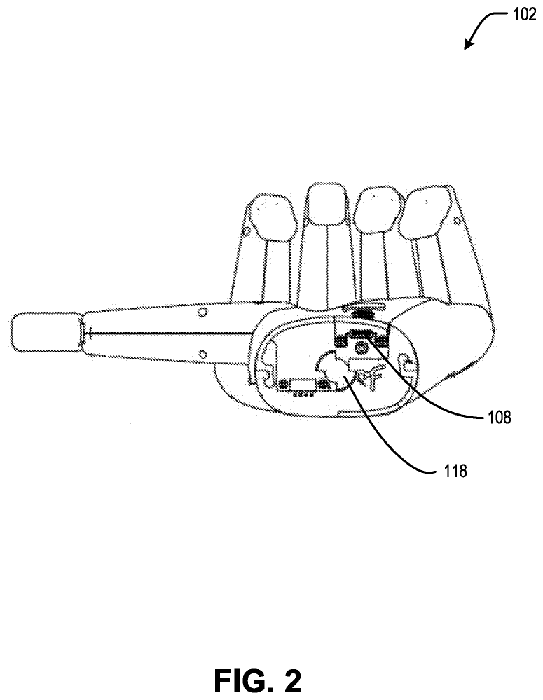

The manner in which the example upper limb prosthetic hand is used for assisting a transradial amputee, along with other aspects, is further explained in details with respect to FIGS. 1-2. It is to be noted that drawings of the present subject matter shown here are for illustrative purposes only and are not to be construed as limiting the scope of the subject matter claimed. Further, the FIGS. 1-2 have been explained together, and same reference numerals have been used for referring identical components and entities.

FIG. 1 illustrates a perspective view of an exemplary upper limb prosthetic hand 100 for assisting a transradial amputee, as per an implementation of the present subject matter. As would be understood, amputee may refer to an individual whose limb has been cut off. Such amputation of the limb may be surgically due to some medical reasons, or may be due to an accident. The proposed upper limb prosthetic hand 100 may efficiently assist the amputee, particularly transradial amputees. The proposed upper limb prosthetic hand 100 may be made of any material which may comply with the medical requirements of prosthetic hands.

As depicted in FIG. 1, the upper limb prosthetic hand 100 may include a terminal device 102. This has also been depicted in FIG. 2. FIG. 2 illustrates a bottom view of an exemplary terminal device to be implemented in an exemplary upper limb prosthetic hand for assisting a transradial amputee, as per an implementation of the present subject matter. The terminal device may be implemented as terminal device 102 implemented in the upper limb prosthetic hand 100, as explained in conjunction with FIG. 1.

In one example, as depicted in FIGS. 1-2, the terminal device 102 may be a palm unit. The terminal device 102, palm unit in the present example, may include a thumb 104 and four fingers 106. Each of the thumb 104 and the four fingers 106 may assist the amputee using the upper limb prosthetic hand 100.

Continuing further, the terminal device 102 may further include a power source (not depicted in FIGS. 1-2). The power source may be configured to power the terminal device 102 and the prosthetic hand 100. In one example, the power source may be at least one of a plurality of Li-ion (Lithium ion) rechargeable batteries. In another example, the batteries in the terminal device may be charged using a USB Type-C charging technique. In such cases, the terminal device 102 may include a receiver 108 for receiving the charging cable. However, it may be noted that all such examples are only illustrative, and any other method or technique known to a person skilled in the art may also be used to power or charge the terminal device 102.

Continuing further, the terminal device 102 may further include a display unit 110 and an input unit 112. The display unit 110 may be implemented as any display unit known to a person skilled in the art. Further, the input unit 112 may be configured to receive one of a touch-based input, a voice-based input, or a combination thereof, from the amputee. Examples of such input unit 112 may include, but are not limited to, a touch-based input, a microphone, or any other device capable of receiving an input from the amputee.

Continuing further, the prosthetic hand 100 may further include a longitudinally extending hollow forearm shell 114 removably coupled to the terminal device 102. The hollow forearm shell 114 may include a coupling means 116. The coupling means 116 may be used to couple the prosthetic hand 100 with a residual stump of the amputee. In one example, the hollow forearm shell 114 may receive a portion of the residual stump of the amputee, and may thereby couple the prosthetic hand 100 with the residual stump of the amputee. In another example, the coupling means 116 may be one of a liner, a socket, or a combination thereof. In another example, the terminal device 102 may include a cavity 118 to receive an extending portion from the hollow forearm shell 114 which may then couple the hollow forearm shell 114 with the terminal device 102. However, it may be noted that all such examples are only illustrative, and any other mechanical coupling mechanism may be used to removably couple the hollow forearm shell 114 with the terminal device 112. All such examples would lie within the scope of the present subject matter.

Continuing further, a plurality of sensors (not depicted in FIGS. 1-2) may be mounted on an inner surface of the hollow forearm shell 114 at the coupling means 116. The plurality of sensors may be positioned in such a manner, that the sensors may be in contact with the residual stump of the amputee. At least one of the plurality of sensors may monitor a muscle movement in the residual stump of the amputee. Based on the monitored muscle movement, the sensors may generate electrical signals. In one example, the plurality of sensors may be surface electromyography (EMG) sensors. In another example, the surface EMG sensors may be made of an origami-inspired flexible material.

Continuing further, the upper limb prosthetic hand 100 may further include a plurality of controllers communicatively coupled to the plurality of sensors and the terminal device 102. The controllers may be implemented as any hardware-based, software-based, or network-based computing devices known to a person skilled in the art. In one example, the controller may be implemented in the terminal device 102 of the prosthetic hand 100. In another example, each of the four fingers 106 of the terminal device 102 may implement an independent controller. However, it may be noted that the prosthetic hand 100 may implement any number of additional controllers at any location. Such examples would also be covered within the scope of the present subject matter.

In operation, based on the generated electrical signals by at least one of the plurality of sensors, the controller may cause at least one of the thumb 104 and the four fingers 106 in the terminal device 102 to move. The reception of the electrical signals and converting them into movements may be done using techniques and mechanisms well known to a person skilled in the art.

In one example, the controller, based on the signals from the sensors, may cause an abduction movement, an adduction movement, a flexion movement, an extension movement, or a combination thereof, in the thumb 104. Such movements, in one example, may be implemented using mechanical components in the thumb which may be known to a person skilled in the art. Examples of such components may include, but are not limited to, thumb bracket, ball bearing, worm gear, and worm. In another example, the thumb 104 may include different actuators for implementing the different movements. In yet another example, one actuator may be used for extension movement and flexion movement, whereas another actuator may be used for abduction movement and adduction movement. In yet another example, the thumb 104 of the prosthetic hand 100 may include a proximal phalange and a distal phalange. Such phalanges may aid in efficient and accurate movements of the thumb 104.

Continuing further, in another example, each of the four fingers 106 may include an independent actuator. In such cases, either the controller may be in communication with each of the four actuators, or each of the fingers 106 may have their respective independent controllers as well. Both such cases, as well as any other examples, would be covered within the scope of the present subject matter.

At least one of the plurality of such controllers, based on the signals from the sensors, may cause an independent movement in each of the fingers 106. Such movements, in one example, may be implemented using mechanical components in each of the four fingers 106 which may be known to a person skilled in the art. Examples of such components may include, but are not limited to, fixed bracket, ball bearing, worm gear, worm, spur gear box, and M2 bolt. All such components may function in conjunction with each other to implement the movement. In another example, each of the four fingers 106 of the prosthetic hand 100 may include a proximal phalange and a distal phalange. Such phalanges may aid in efficient and accurate movements of each of the four fingers 106.

In yet another example, it may be the case that the prosthetic hand 100 may be controlled by a user. In such cases, the user may provide their inputs through the input unit 112. Based on the inputs from the user, the controller may then cause the terminal device 102 to operate. The user may be, in one example, the amputee, or in another example, a physician.

In yet another example, the terminal device 102 may be connected to an external computing device (not shown in FIGS. 1-2). In such cases, in one example, the user may be operating a user interface on such computing devices and thereby control the terminal device 102. It may be noted that all such examples are only illustrative, and the input may be provided to the terminal device 102 in any manner without deviating from the scope of the present subject matter.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

We claim:

1. An upper limb prosthetic hand for transradial amputee, the upper limb prosthetic hand comprising:
a terminal device comprising a thumb and four fingers;
a longitudinally extending hollow forearm shell removably coupled to the terminal device, wherein the hollow forearm shell comprises a coupling means, and wherein the coupling means is to couple the prosthetic hand with a residual stump of the amputee; and
a plurality of sensors mounted on an inner surface of the hollow forearm shell at the coupling means, wherein the plurality of sensors are in contact with the residual stump of the amputee, and wherein at least one of the plurality of sensors is to:
monitor a muscle movement in the residual stump; and
based on the monitored muscle movement in the residual stump, generate electrical signals.

2. The upper limb prosthetic hand as claimed in claim 1, further comprising a plurality of controllers communicatively coupled to the plurality of sensors and the terminal device.

3. The upper limb prosthetic hand as claimed in claim 2, wherein at least one of the plurality of controllers is to:
based on the generated electrical signals by at least one of the plurality of sensors, cause at least one of the thumb and the four fingers in the terminal device to move.

4. The upper limb prosthetic hand as claimed in claim 1, wherein the terminal device is a palm unit.

5. The upper limb prosthetic hand as claimed in claim 1, wherein coupling the prosthetic hand with the residual stump of the amputee comprises the longitudinally extending hollow forearm shell to receive a portion of the residual stump of the amputee.

6. The upper limb prosthetic hand as claimed in claim 1, wherein the coupling means is one of a liner, a socket, or a combination thereof.

7. The upper limb prosthetic hand as claimed in claim 1, wherein the plurality of sensors are surface electromyography (EMG) sensors.

8. The upper limb prosthetic hand as claimed in claim 7, wherein the plurality of surface EMG sensors are made of an origami-inspired flexible material.

9. The upper limb prosthetic hand as claimed in claim 1, wherein the terminal device further comprises a display unit and an input unit.

10. The upper limb prosthetic hand as claimed in claim 9, wherein the input unit is to receive one of a touch-based input, a voice-based input, or a combination thereof, from the amputee.

11. The upper limb prosthetic hand as claimed in claim 1, wherein the terminal device further comprises a power source, wherein the power source is to power the prosthetic hand.

12. The upper limb prosthetic hand as claimed in claim 11, wherein the power source is at least one of a plurality of Li-ion rechargeable batteries.

13. The upper limb prosthetic hand as claimed in claim 2, wherein at least one of the plurality of controllers is implemented in the terminal device.

14. The upper limb prosthetic hand as claimed in claim 2, wherein at least one of the plurality of controllers is independently implemented in each of the four fingers of the terminal device.

15. The upper limb prosthetic hand as claimed in claim 2, wherein each of the four fingers of the terminal device comprises an independent actuator, and wherein the controller, based on the signals from at least one of the plurality of sensors, is to cause each of the four fingers to move independently.

16. The upper limb prosthetic hand as claimed in claim 3, wherein the controller, based on the signals from at least one of the plurality of sensors, is to cause an abduction movement, an adduction movement, a flexion movement, an extension movement, or a combination thereof, in the thumb.

17. The upper limb prosthetic hand as claimed in claim 1, wherein the controller, is to further:
based on an input from a user, cause the terminal device to operate.
